(19) United States
(12) Patent Application Publication
(10) Pub. No.: US 2002/0090363 A1
(43) Pub. Date: Jul. 11, 2002

(54) Sustained bioactive agent delivery device and methods of making and using the same

(76) Inventors: Jonathan David Kurtis, Norwood, MA (US); Robert H. Pierce, Pittsford, NY (US)

Correspondence Address: MINTZ, LEVIN, COHN, FERRIS,, GLOVSKY AND POPEO, P.C., One Financial Center, Boston, MA, 02111, US

(21) Appl. No.: 10/008,340

(22) Filed: Nov. 13, 2001

Related U.S. Application Data
(60) Provisional application No. 60/248,113, filed on Nov. 13, 2000.

(51) Int. Cl. A01K 67/033; A61K 48/00

(52) U.S. Cl. 424/93.21; 800/13

(57) ABSTRACT

Disclosed herein are helminth-based medical devices for sustained delivery of bioactive agents.

### RELATED APPLICATIONS

This application claims priority to U.S. Ser. No. 60/248,113, filed Nov. 13, 2000. The contents of this application are incorporated by reference in their entirety.

### FIELD OF THE INVENTION

The invention relates generally to the field of medical devices, and more particularly, to methods and compositions for sustained delivery of bioactive products.

### BACKGROUND OF THE INVENTION

Delivery of biosynthetic, bioactive molecules into humans and domesticated animals has a broad application in a wide range of therapeutic categories, including the prevention and treatment of neoplastic, metabolic, genetic and infectious diseases. Bioactive products can include, e.g., therapeutically useful polypeptides such as insulin and anti-hemophilic factor. For example, Type I diabetes is known to result from defective glucose metabolism associated with decreased levels of insulin, whereas hemophilia is caused by a lack of the blood protein antihemophilic factor, which is necessary for normal blood to clot. Many disorders are caused by somatic or hereditary genetic mutations which result either in inappropriate expression of a given polypeptide gene product or expression of a defective gene product.

One approach for treating such disorders is gene therapy, in which a nucleic acid encoding a bioactive agent is administered directly to a host or patient. However, significant problems can arise with respect to gene therapy. For example, the local genetic environment for the bioactive-agent encoding nucleic acid, can exert a profound effect on the level of expression of an inserted transgene. Second, uncontrolled insertion of the nucleic acid into the host's genome can lead to insertional mutagenesis causing genetic alterations. The effects of such insertional mutagenesis can potentially lead to cancer. In addition, viral and plasmid vectors are inefficient at targeting specific host cells in the human. In most cases, long lived, non-dividing cells such as liver cells are the target for these approaches. Finally, viral vectors can be immunogenic, resulting in immunologic destruction of the virally transduced cells.

### SUMMARY OF THE INVENTION

The invention is based in part on the discovery of a novel approach for the delivery of bioactive agents to a host. This invention uses genetic manipulation to exploit certain innate characteristics of a group of helminths. The genetically modified helminth worms can be used as improved devices for the sustained delivery of bioactive agents such as polypeptide drugs and other therapeutic substances.

The genetically modified helminths are constructed by transforming a non-pathogenic helminth with foreign nucleic acid, which typically is a DNA encoding a therapeutically useful polypeptide. and then introducing the transformed helminth into a vertebrate host such as, e.g., a human or domesticated animal. Within the parasite, the foreign nucleic acid directs the synthesis of the desired bioactive compound. The helminth then secretes this bioactive agent into the local microenvironment within the host, where it will have its effect. Moreover, a preferred helminth is one that can remain in the host for a desired length of time without causing significant injury to the host. An example of a preferred helminth is one or more transformed male schistosomes.

The drug delivery devices of the invention provide several advantages for delivery of nucleic acids encoding bioactive agents. For example, nucleic acids encoding bioactive agents are introduced first into a helminth host, and transformed helminths in which transgene expression is optimized can be chosen prior to introducing the helminths into the host animal. Thus, the drug delivery devices of invention allows for the local genetic microenvironment of the bioactive agent-encoding nucleic acid to be controlled. In addition, problems associated with alterations in the genome of the host due to the introduction of the transforming nucleic acid are obviated because the nucleic acid is not delivered directly to the host but instead to a helminth carrier that is non-immunogenic in the host and whose association with the host can be controlled. Furthermore, preferred helminths used in the methods of the invention are non-immunogenic in the host and infect appropriate vertebrate hosts with high efficiency.

In one aspect, the invention provides a method for making a sustained drug delivery device. The method includes introducing a nucleic acid encoding a bioactive agent into a female helminth and selecting a female transformed with the bioactive agent-encoding nucleic acid. The transformed female is then crossed to at least one non-transformed male helminth and a progeny male containing the stably transformed nucleic acid is isolated, thereby making a sustained drug delivery device. The female helminth is preferably stably transformed with the bioactive agent-encoding nucleic acid.

Also provided by the invention is a sustained drug delivery device that includes a stably transformed helminth male prepared according to the above-described method. In some embodiments, the sustained delivery device is provided as a sustained portal delivery device that includes a stably transformed male helminth located in fluid communication with the host's portal blood stream. The drug delivery device can be provided if desired along with a pharmaceutically acceptable carrier.

The host can be, e.g., any metazoan in which the helminth can propagate. Suitable hosts include, e.g. vertebrates such as birds or mammals. A preferred mammals include, e.g. a human, non-human primate, cow, pig, horse, dog or cat.

Also provided by the invention is a method of delivering a bioactive agent to a host by introducing a stably transformed male helminth into a host, wherein the male is stably transformed with a nucleic acid encoding the bioactive agent, and wherein expression of the nucleic acid in the helminth results in delivery of the bioactive agent to the host. The host can be, e.g., any metazoan in which the helminth can propagate. Suitable hosts include, e.g. vertebrates such as birds or mammals. A preferred mammals include, e.g. a human, non-human primate, cow, pig, horse, dog or cat. The invention also provides a miracidia containing the helminth-containing sustained drug delivery device, as well as a snail that includes this miracidia. Also featured by the invention is a cercaria that includes the the helminth-containing sustained drug delivery device described herein.

Also within the invention is a method of treating or preventing a disease in a host. The method includes introducing a stably transformed helminth male into a host, wherein the male is stably transformed with a nucleic acid encoding a bioactive agent, wherein expression of the nucleic acid in the helminth results in delivery of the bioactive agent to the host in an amount sufficient to treat or prevent the disease. The disease can be, e.g., diabetes mellitus type 1, hemophilia, dwarfism, Gaucher's disease, alpha₁-antitrypsin deficiency, inflammatory bowel disease or growth acceleration in cattle.

The helminth used can be, e.g., a hookworm, roundworm, pinworm or tapeworm. A preferred helminth is a Schistosome species. Examples of suitable Schistosome species include Schistosoma mansoni, Schistosoma japonicum, or Schistosoma hematobium.

The nucleic acid encoding the bioactive agent can be DNA (e.g., a cDNA) or RNA. The bioactive agent can be, e.g., a stable RNA or a polypeptide. When the polypeptide is a secreted polypeptide, the polypeptide is preferably a secreted polypeptide and/or a post-translationally modified polypeptide. Examples of post-translational modifications include, e.g., glycosylation. Examples of suitable polypeptides include, e.g. a cytokine, enzyme, hormone, or neurotransmitter.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

Helminth-based drug delivery devices are constructed by transforming a suitable helminth host with foreign DNA. The transformed helminths are then introduced into a desired host. Within the transformed helminth host, the foreign DNA directs the synthesis of a desired bioactive compound, which is typically a polypeptide. The transformed helminth will then secrete this bioactive agent into the appropriate microenvironment within the host where it will have its effect.

A suitable helminth is one that is preferably non-toxic in the host in which it will be introduced. The helminth is in addition preferably selected so that its presence in the host can be modulated. For example, a preferred helminth is a transformed helminth that can be readily removed by treating the host with an antibiotic or other agent that removes the transformed helminth.

Suitable helminths for use in human hosts include, e.g., Hookworms (Ancylostoma duodenale and Necator americanus), Roundworms (Ascaris lumbricoides), Whipworms (Trichuris trichiura), Pinworms (Enterobius vermicularis), Tapeworms (Taenia saginata), and lung flukes (Paragonimus westermani). Modified protozoan malarial organisms (for example, liver-stage restricted hypnozoites) that exhibit intrahepatocellular niches are particularly preferred.

Another preferred helminth for use in the devices and methods of the invention is a schistosome. The liver flukes (Schistosoma mansoni, Schistosoma japonicum, and Schistosoma hematobium) are particularly preferred targets for the development of a parasite based protein/drug delivery system. In unisexual male infections, Schistosoma spp. can live for extended periods of time within the host without causing disease.

Three of the major species that infect humans are S. mansoni, S. japonicum and S. haematobium.

Adult schistosomes typically dwell as pairs of males and females in veins of their definitive host. Various Schistosoma species localize to specific organs: S. mansoni in the portal veins draining the intestine, S. japonicum in the veins of the small intestine and S. haematobium in the urinary bladder plexus.

The drug delivery devices of the invention exploit distinctive aspects of the helminth life cycle. For example, Schistosoma mansoni are dioecious trematodes (flatworms), which live and reproduce in the veins of the abdominal mesenteric plexus (between the gut and the liver). When coupled with a male, female S. mansoni worms are capable of producing several hundred eggs per day, many of which enter the intestinal lumen and pass out of the hosts body with the feces. Those eggs which are not excreted induce a chronic inflammatory response, which surrounds the trapped eggs.

When eggs exit the body and enter fresh water, they hatch, releasing free swimming miracidia. To continue the life cycle, miracidia infect a particular species of snail (Biomphalari) within 6-12 hours after hatching. Free swimming, forked tailed cercariae emerge from the infected snail after 4 weeks of asexual reproduction (sporocyst stages). Within 1-2 days, before their glycogen reserves are depleted, the cercariae must infect their definitive host by penetrating intact, exposed skin. Upon penetration, the cercariae loose their tails and undergo extensive physiologic changes, including loss of their glycocalyx, a thick protective proteoglycan layer. Following penetration, parasites form a unique heptalaminar surface membrane and develop into schistosomula. Schistosomula migrate through dermal tissues for approximately 3 days before entering the bloodstream. Parasites travel in the direction of blood flow and must pass through a lung stage before giving rise to adult worms 5-6 weeks post infection. These adult worms live in the abdominal mesenteric plexus, thus completing the lifecycle. During this migration phase the host has no clinical symptoms.

The schistosomes possess several desirable properties for use as drug delivery devices according to the invetnion. First, adult male schistosome infections cause no disease. After stably infecting their host, adult male schistosomes are localize to the portal blood stream, where they can function as sensors of the constitutive and nutritional state of the subject (e.g., human subject). Second, adult male schistosome can live for many years in an infected host. In addition, adult male schistosomes are impervious to immune attack. A further advantage of using schistosomes is that these organisms can be eradicated from the host with a single dose of a safe oral medicine. Further, juvenile male schistosomes can be cryopreserved indefinitely for subsequent use. Also, adult male schistosomes proteins that demonstrate post-translational modifications (such as glycosylation) that are typical of higher eukaryotes.

Generation of Stably Transformed Female Helminths Encoding Bioactive Agents

Helminths can be maintained by utilizing worm culture techniques known in the art. For example, schistosomes can be cultured as described in Basch et al., J. Parasitol. 69:567-69, 1983.

Nucleic acids encoding bioactive constructs can similarly be made using methods known in the art. If desired, a nucleic acid encoding a bioactive polypeptide can be constructed containing expression control sequences (such as promoters, enhancers and the like) that optimize expression of the bioactive-agent encoding nucleic acid in the helminth host. The construct encoding the bioactive agent is typically provided as a vector, e.g., in a plasmid or viral vector. The vector is preferably an expression control vector. A preferred viral factor is a recombinant adeno-associated virus, which is known to transduce a wide variety of cell types (Robbins et al., Trends in Biotechnology 16:35-40). Nucleic acids encoding the bioactive agent can optionally be provided flanked by sequence that facilitate integration into chromosomal DNA sequences. An example of these types of sequences is the inverse terminal repeat (ITR) sequences of adeno-associated virus (Pieroni et al., Virology 249:249-59, 1998).

The nucleic acid encoding the bioactive agent is preferably introduced into the germline of a female helminth (e.g., a female schistosome). Preferably, the nucleic acid is stably integrated into the germline of the helminth.

Several methods of transformation are known in the art. In one preferred embodiment, transformation is performed by microinjecting naked plasmid DNA into the female's ovary. Microinjection can be performed using microinjection transformation techniques developed for germline transformation of the nematode Caenorhabditis elegans (Fire et al., EMBO J.8:3419-28, 1989) Other methods for transformation include particle bombardment (Davis et al., Proc. Natl. Acad. Sci. USA 96:8687-92, 1999; Unnasch et al., Transfection of Brugia Malayi Division of Geographic Medicine, University of Alabama at Birmingham, Ala.; and Biological Science s, Fordham University. Abstract #203. American Society of Tropical Medicine and Hygiene, 49^{th} Annual Meeting, Houston, Texas, 2000) liposome mediated transformation, electroporation. If desired, the transforming nucleic acid can include a selectable marker that facilitates selection of the bioactive agent-encoding nucleic acid.

The presence of foreign nucleic acid in a female adult helminth can be confirmed using methods known in the art, e.g., by using polymerase chain reaction (PCR) amplification to detect the introduced DNA.

A transformed female is mated with a wild type male schistosome using standard worm culture techniques known to those skilled in the art. F1 hybrid offspring are identified and propagated. Techniques for performing genetic manipulations and propagation in helminths are described in, e.g., Newport et al., 84:481-90, 1982; Kawanaka et al., J. Parasitol. 71:368-70, 1985; Yoshino et al. J. Parasitol. 81:714-22, 1995; DiConza et al., J. Parasitol. 58:181-82, 1972; Jordane, Annales de Parasitologie Humaine et Comparee 59:361-67, 1984; Cohen et al., Exp. Parasitol. 57:15-19; and Cohen et al., J. Parasitol.: 74:963-69). The clonally propagated offspring and the F1 adults are screened for the presence of the transgene using art-recognized methods. For example, screening can be accomplished by PCR and fluorescent based technologies in the case of vectors encoding Green Fluorescent Protein (GFP).

In some embodiments, a progeny male carrying the bioactive agent-encoding nucleic acid is used to inject a desired host using standard techniques. For example, a mammalian host can be infected with a transformed male using techniques described in Purnell, Annals of Tropical Med.& Parasitol.:74:963-69, 1988.

In other embodiments, a transformed female that does not cause disease in the host is used. Preferably, the transformed female is sterile (e.g., does not lay eggs in the host). In some preferred embodiments, the transformed female can live for a desired length of time in the host in the absence of a male.

Transformed helminths can be injected into any suitable hosts. In general, the host will be a metazoan and is preferably a vertebrate such as a reptile, bird, or mammal. Particularly preferred hosts include humans, non-human primates, and domesticated animals, including dogs, cats, horses, cows, pigs, and sheep. The helminth is selected to that it can produce the appropriate association with the desired host. For example, some helminths exhibit a wide host range. For example, adult male schistosomes infect laboratory rodents and non-human primates. In addition, S. japonicum infects a broad range of domesticated animals including cattle, thus expanding potential applications. Thus, the present invention provides a method for introduction of the transformed male schistosome into human and domesticated animal host.

Bioactive Agents

As used herein, the term “bioactive agent” refers to a compound that exerts an effect on a living organism. Stable RNA molecules, peptides, and proteins (including cytokines, enzymes, hormones or neurotransmitters) are among the examples of bioactive agents. Substances such as insulin, gamma interferon, bone morphogenetic proteins, tissue plasminogen activator, beta interferon and Factor VIII are among the preferred art-recognized substances of current therapeutic interest. Other currently preferred substances would include those suitable for treating the following selected diseases such as, but not limited to, osteoporosis, diabetes, cancer, severe anemia, short stature and hemophilia.

Bioactive agents suitable for use in the devices and methods of the invention can be conveniently divided intoare two broad classes, termed class I and class II targets. For class I targets, the exact amount of bioactive agent delivered into the blood stream does not require precise control; therefore, its delivery into the blood stream is loosely controlled by adjusting the number of parasites in the human host. Some examples of class I targets include: hemophilia—by infecting a host with a sustained delivery device that constitutively expresses Clotting Factor VIII or IX; dwarfism—by infecting a host with a sustained delivery device that constitutively expresses human growth hormone and eradicate the device when the child achieves normal height; Gaucher's Disease—by infecting a host with sustained delivery device that constitutively expresses glucocerebrosidase; alpha₁-antitrypsin deficiency—by infecting a host with a sustained delivery device that constitutively expresses alpha₁-antitrypsin; inflammatory bowel disease—by infecting a host with a sustained delivery device that secretes anti-TNF antibody into the gut lumen; and growth acceleration of cattle—by infecting a host with a sustained delivery device that expresses bovine growth hormone.

For class II targets, the diseases and conditions treated with bioactive agents require tight and continuous regulation of biotherapeutic output. For class II targets, the amount of bioactive agent produced by the sustained delivery device is tightly regulated. The helminth preferably can modulate the production of the bioactive agent in response to environmental signals within the subject. The prototypic class II target is diabetes mellitus type 1, in which loss of pancreatic islet cells results in the inability of the patient to make and secrete insulin into the blood in response to blood glucose concentrations. In a preferred embodiments, a patient, e.g., a diabetic patient, is infected with a helminth engineered to secrete insulin in a tightly-regulated response to blood glucose levels.

In this embodiment, the cDNA for insulin is placed under the control of a glucose responsive element. Schistosomes have sensors for a variety of external signals including steroid hormones (Giannini et al., Parasitol. 110(Pt.2):155, 1995; Rumjanek et al., Mem Inst Oswaldo Cruz. 1:197, 1989), 2), presence of males (Grevelding et al., Parasitol. 115(Pt.6):635, 1997), and N-acetyl cysteine (Khalife et al., Parasitol. 111(Pt.4):469, 1995). In response to these signals, the worms turn on expression of specific genes via transcription factors. These transcription factors bind to response elements that control the expression of the responsive genes.

A preferred responsive element or elements is a glucose-responsive element. Schistosomes express several glucose transporters on their surface (Skelly and Shoemaker, Proc Natl Acad Sci USA. 93:3642, 1996). Thus, the expression control sequences (such as promoters and enhancers) of these and other schistosomal genes up-regulated by the presence of glucose can be operably linked to a nucleic acid encoding human insulin (e.g., an human insulin cDNA). The shistosome is transformed with this construct, and, following introduction into a host (e.g., a diabetic patient), the transformed schistosome secretes insulin in proportion to the glucose concentration in its environment.

Additional embodiments are within the claims.

What is claimed is:

1. A method for making a sustained drug delivery device, the method comprising
introducing a nucleic acid encoding a bioactive agent into a female helminth;
selecting a female stably transformed with said bioactive agent-encoding nucleic acid;
crossing said stably transformed female to a non-transformed male helminth; and
isolating a progeny male containing said stably transformed nucleic acid, thereby making a sustained drug delivery device.

2. The method of claim 1, wherein the helminth is a hookworm, roundworm, pinworm or tapeworm.

3. The method of claim 1, wherein the helminth is a Schistosome species.

4. The method of claim 3, wherein the Schistosome species is Schistosoma mansoni, Schistosoma japonicum, or Schistosoma hematobium.

5. The method of claim 1, wherein the bioactive agent is a polypeptide.

6. The method of claim 4, wherein the polypeptide is a secreted polypeptide.

7. The method of claim 4, wherein the polypeptide is a post-translationally modified polypeptide.

8. The method of claim 7, wherein the post-translational modification is glycosylation.

9. The method of claim 5, wherein the polypeptide is a cytokine, enzyme, hormone, or neurotransmitter.

10. A sustained drug delivery device comprising a stably transformed helminth male prepared according to the method of claim 1.

11. The sustained delivery device of claim 10, wherein the helminth is a Schistosome species.

12. A miracidia containing the sustained drug delivery device of claim 11.

13. A snail containing the miracidia of claim 12.

14. A cercaria comprising the sustained drug delivery device of claim 11.

15. A pharmaceutical composition comprising the sustained delivery device of claim 11 and a pharmaceutically acceptable carrier.

16. A method of delivering a bioactive agent to a host, the method comprising
introducing a stably transformed male helminth into a host, wherein said male is stably transformed with a nucleic acid encoding said bioactive agent, wherein expression of said nucleic acid in said helminth results in delivery of said bioactive agent to said host.

17. The method of claim 16, wherein the host is a human or domesticated animal.

18. A method of treating or preventing a disease in a host, the method comprising introducing a stably transformed helminth male into a host, wherein said male is stably transformed with a nucleic acid encoding a bioactive agent, wherein expression of said nucleic acid in said helminth results in delivery of said bioactive agent to said host in an amount sufficient to treat or prevent said disease.

19. The method of claim 18, wherein the disease is diabetes mellitus type 1, hemophilia, dwarfism, Gaucher's disease, alpha₁-antitrypsin deficiency, inflammatory bowel disease or growth acceleration in cattle.

20. The method of claim 19, wherein the host is a human.
